(19) United States
(12) Patent Application Publication
(10) Pub. No.: US 2024/0156604 A1
(43) Pub. Date: May 16, 2024

(54) Mesh Reinforced Polyurethane Synthetic Meniscus

(71) Applicant: Arthrex, Inc., Naples, FL (US)

(72) Inventors: Patrick A. Smith, Naples, FL (US); Reinhold Schmieding, Naples, FL (US); Andrew Osika, Naples, FL (US)

(21) Appl. No.: 18/507,460

(22) Filed: Nov. 13, 2023

Related U.S. Application Data
(60) Provisional application No. 63/425,278, filed on Nov. 14, 2022.

(51) Int. Cl. A61F 2/30; A61L 27/18; A61L 27/34; A61L 27/48; A61L 27/56

(52) U.S. Cl. CPC A61F 2/30756; A61L 27/18; A61L 27/34; A61L 27/48; A61L 27/56; A61F 2002/0068; A61F 2002/30131

(57) ABSTRACT

The present disclosure provides a prosthetic device for replacing or augmenting a damaged meniscus.

### RELATED APPLICATIONS

This application claims priority to U.S. Provisional Application No. 63/425,278 entitled “Mesh Reinforced Polyurethane Synthetic Meniscus,” filed on Nov. 14, 2022, the contents of which are hereby incorporated by reference in its entirety.

### BACKGROUND

The present disclosure generally relates to medical prosthetic devices, systems, and methods. More specifically, in some instances the present disclosure relates to prosthetic devices that replace at least part of the functionality of the natural meniscus. Each knee has two menisci, a lateral meniscus and a medial meniscus. Each meniscus is a crescent-shaped fibrocartilaginous tissue attached to the tibia at an anterior and a posterior horn. Damage to the meniscus can cause pain and arthritis. Disclosed herein is a prosthetic device for replacing or augmenting a damaged meniscus.

### SUMMARY

The disclosure herein includes a prosthetic device for replacing or augmenting a damaged meniscus. The prosthetic device described herein is reinforced with a mesh material throughout at least a portion of a polyurethane body, and includes exposed mesh material for anterior and posterior root fixation.

In particular, in one aspect, the present disclosure provides a prosthetic device for replacing a damaged meniscus including a polyurethane body having a first end and a second end opposite the first end. The polyurethane body includes a first curved surface extending between the first end and the second end, and a second curved surface extending between the first end and the second end. The prosthetic device further includes a mesh positioned within at least a portion of the polyurethane body. The mesh includes a first plurality of strands extending from the first end of the polyurethane body, a second plurality of strands extending from the second end of the polyurethane body, and a third plurality of strands extending from the first curved surface of the polyurethane body.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is a perspective view of an example prosthetic device for replacing a damaged meniscus.

FIG. 2 is a cross-sectional view along line A-A of the example prosthetic device of FIG. 1.

### DETAILED DESCRIPTION

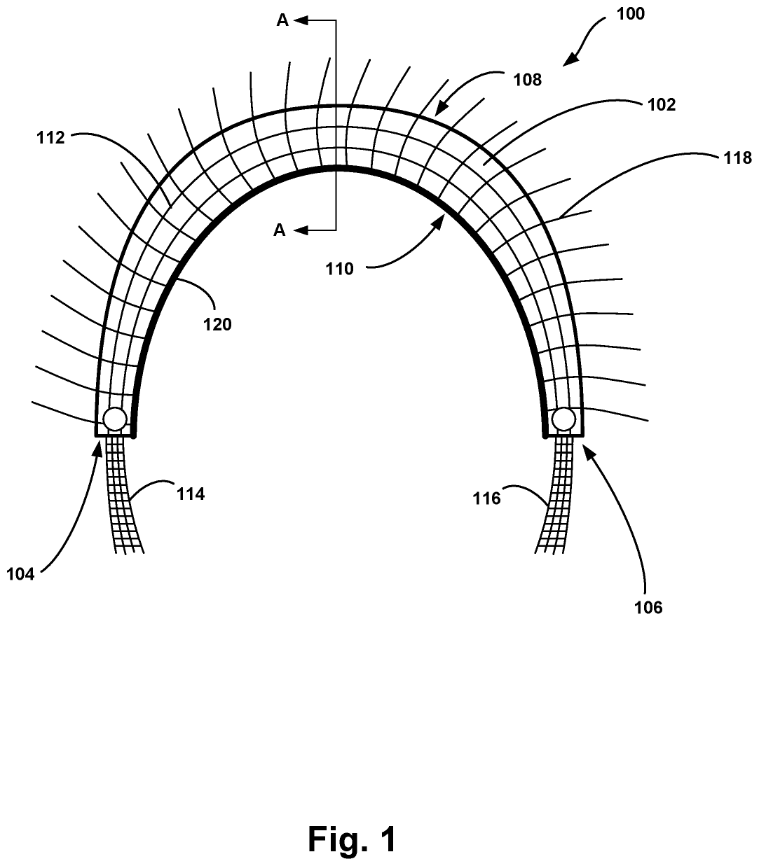

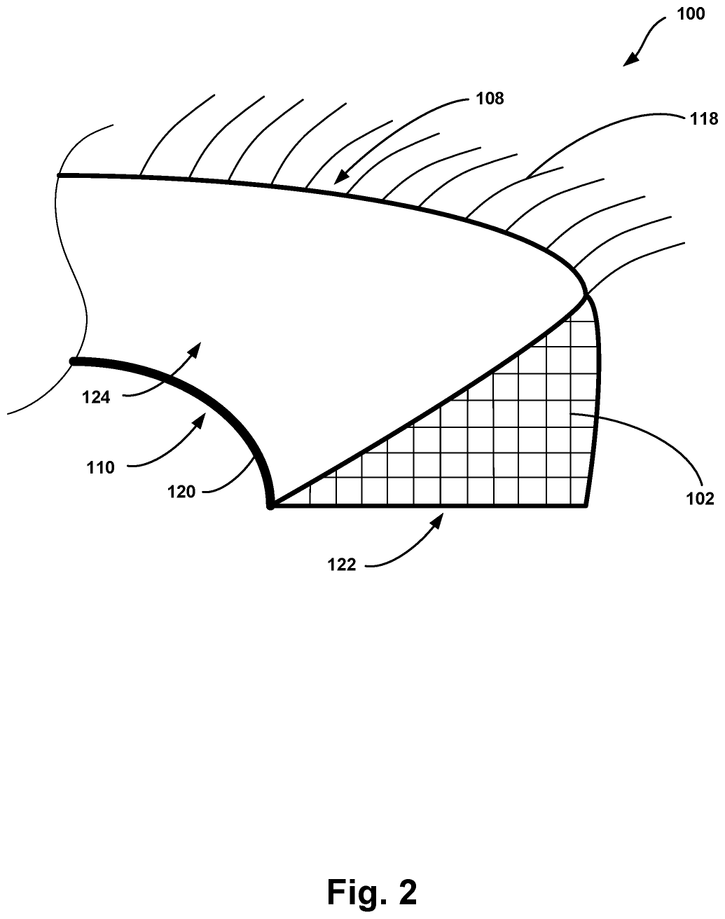

With reference to the Figures, FIGS. 1-2 illustrate a prosthetic device 100 for replacing a damaged meniscus. In particular, the prosthetic device 100 for replacing a damaged meniscus shown in FIGS. 1-2 comprises a polyurethane body 102 having a first end 104 and a second end 106 opposite the first end 104. The polyurethane body 102 includes a first curved surface 108 extending between the first end 104 and the second end 106. The polyurethane body further includes a second curved surface 110 extending between the first end 104 and the second end 106. The prosthetic device 100 further includes a mesh 112 positioned within at least a portion of the polyurethane body 102. The mesh 112 includes a first plurality of strands 114 extending from the first end 104 of the polyurethane body 102, a second plurality of strands 116 extending from the second end 106 of the polyurethane body 102, and a third plurality of strands 118 extending from the first curved surface 108 of the polyurethane body 102. Each of the first plurality of strands 114, the second plurality of strands 116, and the third plurality of strands 118 may be interconnected to the mesh 112 embedded within the polyurethane body 102.

In one example, a radius of curvature of the first curved surface 108 of the polyurethane body 102 is greater than a radius of curvature of the second curved surface 110 of the polyurethane body 102. As such, as shown in FIG. 1, the polyurethane body 102 may have a semicircular shape. In such an example, the first plurality of strands 114 extending from the first end 104 of the polyurethane body 102 and the second plurality of strands 116 extending from the second end 106 of the polyurethane body 102 may be substantially parallel to one another.

In one example, the prosthetic device 100 further includes a porous material 120 coupled to at least a portion of the second curved surface 110 of the polyurethane body 102. In one example, the porous material 120 is chemically bonded to at least a portion of the second curved surface 110 of the polyurethane body 102. The porous material 120 may comprise a non-resorbable, reticulated, cross-linked, polycarbonate polyurethane-urea matrix, as a non-limiting example. The porous material 120 may help support fibrovascular tissue ingrowth after implatation of the prosthetic device 100.

In one example, a density of the first plurality of strands 114 and a density of the second plurality of strands 116 are substantially equal to one another. In another example, a density of the first plurality of strands 114 and a density of the second plurality of strands 116 are greater than a density of the third plurality of strands. In one example, the density of a given plurality of strands may be determined by a number of strands per given area.

In one example, as shown in FIG. 2, a bottom surface 122 of the polyurethane body 102 may be substantially flat and a top surface 124 of the polyurethane body 102 may be a concave sloped surface. As such, a cross-section of the polyurethane body 102 long the line A-A shown in FIG. 1 results in a substantially triangular shape. Further, a thickness of the first curved surface 108 of the polyurethane body 102 is greater than a thickness of the second curved surface 110 of the polyurethane body 102.

In one example, the mesh 112 comprises a multi-strand, long chain ultra-high molecular weight polyethylene (UHMWPE) core with a braided jacket of polyester and UHMWPE.

In one example, the polyurethane body 102 comprises a thermoplastic polycarbonate polyurethane (PCU).

In use, the mesh 112 positioned within at least a portion of the polyurethane body 102 provides increased structural strength to the prosthetic device 100. The first plurality of strands 114 extending from the first end 104 of the polyurethane body 102 and the second plurality of strands 116 extending from the second end 106 of the polyurethane body 102 may be used for root fixation of the prosthetic device 100 to the patient. The third plurality of strands 118 extending from the first curved surface 108 of the polyurethane body 102 may be used for peripheral and capsular fixation of the prosthetic device 100 to the patient. The porous material 120 may be positioned in contact with the bone of the patient, and may help support fibrovascular tissue ingrowth.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and examples have been disclosed herein, other aspects and examples will be apparent to those skilled in the art. The various aspects and examples disclosed herein are for purposes of illustration and are not intended to be limiting, with the true scope being indicated by the following claims, along with the full scope of equivalents to which such claims are entitled. It is also to be understood that the terminology used herein is for the purpose of describing particular examples only, and is not intended to be limiting.

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” or “an example” means that one or more feature, structure, or characteristic described in connection with the example is included in at least one implementation. The phrases “one embodiment” or “one example” or “an example” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The limitations of the following claims are not written in means-plus-function format and are not intended to be interpreted based on 35 U.S.C. § 112(f), unless and until such claim limitations expressly use the phrase “means for” followed by a statement of function void of further structure.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, in one embodiment, the terms “about,” “approximately,” or “substantially” can refer to ±5% of a given value.

Illustrative, non-exhaustive examples, which may or may not be claimed, of the subject matter according the present disclosure are provided below.

What is claimed is:

1. A prosthetic device for replacing a damaged meniscus, the prosthetic device comprising:
a polyurethane body having a first end and a second end opposite the first end, wherein the polyurethane body includes a first curved surface extending between the first end and the second end, and wherein the polyurethane body includes a second curved surface extending between the first end and the second end; and
a mesh positioned within at least a portion of the polyurethane body, wherein the mesh includes a first plurality of strands extending from the first end of the polyurethane body, wherein the mesh includes a second plurality of strands extending from the second end of the polyurethane body, and wherein the mesh includes a third plurality of strands extending from the first curved surface of the polyurethane body.

2. The prosthetic device of claim 1, wherein of the first plurality of strands, the second plurality of strands, and the third plurality of strands are interconnected to the mesh embedded within the polyurethane body.

3. The prosthetic device of claim 1, wherein a radius of curvature of the first curved surface of the polyurethane body is greater than a radius of curvature of the second curved surface of the polyurethane body.

4. The prosthetic device of claim 3, wherein the polyurethane body is semicircular in shape.

5. The prosthetic device of claim 4, wherein the first plurality of strands extending from the first end of the polyurethane body and the second plurality of strands extending from the second end of the polyurethane body are substantially parallel to one another.

6. The prosthetic device of claim 1, wherein a density of the second plurality of strands and a density of the second plurality of strands are substantially equal to one another.

7. The prosthetic device of claim 6, wherein the density of the second plurality of strands and the density of the second plurality of strands are greater than a density of the third plurality of strands.

8. The prosthetic device of claim 1, wherein a bottom surface of the polyurethane body is be substantially flat and a top surface of the polyurethane body comprises a concave sloped surface.

9. The prosthetic device of claim 1, wherein a thickness of the first curved surface of the polyurethane body is greater than a thickness of the second curved surface 110 of the polyurethane body.

10. The prosthetic device of claim 1, wherein the mesh comprises a multi-strand, long chain ultra-high molecular weight polyethylene (UHMWPE) core with a braided jacket of polyester and UHMWPE.

11. The prosthetic device of claim 1, wherein the polyurethane body comprises a thermoplastic polycarbonate polyurethane (PCU).

12. The prosthetic device of claim 1, further comprising:
a porous material coupled to at least a portion of the second curved surface of the polyurethane body.

13. The prosthetic device of claim 12, wherein the porous material is chemically bonded to at least a portion of the second curved surface of the polyurethane body.

14. The prosthetic device of claim 12, wherein the porous material comprises a non-resorbable, reticulated, cross-linked, polycarbonate polyurethane-urea matrix.
